(19) United States
(12) Patent Application Publication
(10) Pub. No.: US 2008/0269882 A1
(43) Pub. Date: Oct. 30, 2008

(54) INTRAOCULAR LENS WITH ASYMMETRIC OPTICS

(75) Inventors: Michael J. Simpson, Arlington, TX (US); Xiaoxiao Zhang, Forth Worth, TX (US)

Correspondence Address: ALCON, IP LEGAL, TB4-8, 6201 SOUTH FREEWAY, FORT WORTH, TX, 76134, US

(73) Assignee: ALCON UNIVERSAL LTD., Fort Worth, TX (US)

(21) Appl. No.: 11/742,035

(22) Filed: Apr. 30, 2007

(51) Int. Cl. A61F 2/16

(52) U.S. Cl. 623/6.17

(57) ABSTRACT

In one aspect, an intraocular lens (IOL) is discloses that includes an optic having a central portion and a peripheral extension that partially surrounds the central portion. Once implanted in a patient's eye, the IOL's optic forms an image of a field of view with the peripheral extension inhibiting dysphotopsia. While in some embodiments, the peripheral extension provides focusing of light incident thereon, in other embodiments, it can include at least one textured surface for scattering the light or at least one opaque surface for preventing the light from reaching the retina. In other embodiments, the peripheral extension can include one or more translucent surface(s) for diffusing the light passing therethrough to inhibit dysphotopsia.

### RELATED APPLICATIONS

This application is related to the following co-pending applications concurrently filed herewith, each of which is herein incorporated by reference: “Intraocular Lens With Peripheral Region Designed to Reduce Negative Dysphotopsia,” (Attorney Docket No. 2817), “IOL Peripheral Surface Designs to Reduce Negative Dysphotopsia,” (Attorney Docket No. 3345), “Intraocular Lens with Asymmetric Haptics,” (Attorney Docket No. 3227), “Intraocular Lens With Edge Modification,” (Attorney Docket No. 3225), “A New Ocular Implant to Correct Dysphotopsia, Glare, Halo, and Dark Shadow,” (Attorney Docket No. 3226), “Haptic Junction Designs to Reduce Negative Dysphotopsia,” (Attorney Docket No. 3344), and “Graduated Blue Filtering Intraocular Lens,” (Attorney Docket No. 2962).

### BACKGROUND

The present invention relates generally to intraocular lenses (IOLs), and particularly to IOLs that provide a patient with an image of a field of view without the perception of visual artifacts in the peripheral visual field.

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses are routinely employed to replace such a clouded natural lens. Although such IOLs can substantially restore the quality of a patient's vision, some patients with implanted IOLs report aberrant optical phenomena, such as halos, glare or dark regions in their vision. These aberrations are often referred to as “dysphotopsia.” In particular, some patients report the perception of dark shadows, particularly in their temporal peripheral visual fields. This phenomenon is generally referred to as “negative dysphotopsia.”

Accordingly, there is a need for enhanced IOLs, especially IOLs that can reduce dysphotopsia, in general, and the perception of dark shadows or negative dysphotopsia, in particular.

### SUMMARY

The present invention generally provides asymmetric intraocular lenses (IOLs) with asymmetric optics that alleviate, and preferably eliminate, the perception of dark shadows that some IOL patients report.

The present invention is based, in part, on the discovery that the shadows perceived by IOL patients can be caused by a double imaging effect when light enters the eye at very large visual angles. More specifically, it has been discovered that in many conventional IOLs, most of the light entering the eye is focused by both the cornea and the IOL onto the retina, but some of the peripheral light misses the IOL and it is hence focused only by the cornea. This leads to the formation of a second peripheral image. Although this image can be valuable since it extends the peripheral visual field, in some IOL users it can result in the perception of a shadow-like phenomenon that can be distracting.

To reduce the potential complications of cataract surgery, designers of modern IOLs have sought to make the optical component (the “optic”) smaller (and preferably foldable) so that it can be inserted into the capsular bag with greater ease following the removal of the patient's natural crystalline lens. The reduced lens diameter, and foldable lens materials, are important factors in the success of modern IOL surgery, since they reduce the size of the corneal incision that is required. This in turn results in a reduction in corneal aberrations from the surgical incision, since often no suturing is required. The use of self-sealing incisions results in rapid rehabilitation and further reductions in induced aberrations. However, a consequence of the optic diameter choice is that the IOL optic may not always be large enough (or may be too far displaced from the iris) to receive all of the light entering the eye.

Moreover, the use of enhanced polymeric materials and other advances in IOL technology have led to a substantial reduction in capsular opacification, which has historically occurred after the implantation of an IOL in the eye, e.g., due to cell growth. Surgical techniques have also improved along with the lens designs, and biological material that used to affect light near the edge of an IOL, and in the region surrounding the IOL, no longer does so. These improvements have resulted in a better peripheral vision, as well as a better foveal vision, for the IOL users. Though a peripheral image is not seen as sharply as a central (axial) image, peripheral vision can be very valuable. For example, peripheral vision can alert IOL users to the presence of an object in their field of view, in response to which they can turn to obtain a sharper image of the object. It is interesting to note in this regard that the retina is a highly curved optical sensor, and hence can potentially provide better off-axis detection capabilities than comparable flat photosensors. In fact, though not widely appreciated, peripheral retinal sensors for visual angles greater than about 60 degrees are located in the anterior portion of the eye, and are generally oriented toward the rear of the eye. In some IOL users, however, the enhanced peripheral vision can lead to, or exacerbate, the perception of peripheral visual artifacts, e.g., in the form of shadows.

Dysphotopsia (or negative dysphotopsia) is often observed by patients in only a portion of their field of vision because the nose, cheek and brow block most high angle peripheral light rays—except those entering the eye from the temporal direction. Moreover, because the IOL is typically designed to be affixed by haptics to the interior of the capsular bag, errors in fixation or any asymmetry in the bag itself can exacerbate the problem—especially if the misalignment causes more peripheral temporal light to bypass the IOL optic.

In many embodiments of the invention, the IOL's optic is extended asymmetrically in the nasal direction to receive peripheral light rays entering the eye from the temple side (herein referred to as temporal peripheral rays) at large visual angles and to capture and/or redirect those rays so as to eliminate the perception of dark shadows by the IOL user. In some cases, this is achieved by ensuring that those rays would not form a second peripheral image. More preferably, rather than inhibiting the formation of the second peripheral image, some of the light rays are directed, e.g., via scattering, to a shadow region between a primary image, formed by the IOL, and a secondary peripheral image formed by light rays that miss the IOL, so as to inhibit dyphotopsia while preserving the second peripheral image—albeit in an attenuated form. Such redirecting of the peripheral light rays allows the IOL user to enjoy the expanded peripheral vision provided by the second peripheral image without the perception of visual artifacts due to dysphotopsia.

In one aspect, an intraocular lens (IOL) is disclosed that includes an optic having a central portion and a peripheral extension that partially surrounds the central portion. Once the IOL is implanted in the eye, the optic forms an image of a field of view with the peripheral extension inhibiting (i.e., ameliorating and preferably preventing) the perception of visual artifacts in the patient's peripheral vision. For example, the peripheral extension can inhibit the formation of a secondary image by peripheral light rays entering the eye at large visual angles or can redirect some light rays to a shadow region between such a secondary image and an image formed by the central portion. In other words, the peripheral extension can inhibit dysphotopsia.

In a related aspect, the peripheral extension is formed as a contiguous optical structure that is asymmetrically disposed relative to the optic's central portion.

In related aspects, the peripheral extension can provide focusing of light incident thereon onto the retina such that, together with the light focused by the central portion, a single image of a field of view can be formed. Alternatively, the peripheral extension can include at least one textured surface that inhibits the peripheral rays incident thereon from forming a secondary image on the retina or to cause some of the peripheral light rays to be directed to a shadow region between an image formed by the IOL and a second peripheral image formed by rays that miss the IOL and are refracted only by the cornea onto the retina. In other embodiments, the peripheral extension can be opaque or translucent so as to inhibit dysphotopsia. In other cases, the peripheral extension can include a diffractive structure or a Fresnel lens.

The IOL's optic can include an anterior surface and a posterior surface, each of which is characterized by a central surface portion and a peripheral surface extension that partially surrounds the central portion. While in some embodiments, the central portion and the peripheral extension of each surface form a contiguous optical surface, in other embodiments, they can be formed as separate surfaces that are coupled to one another. In many embodiments, the peripheral extension of the anterior surface is adapted to receive at least some of the peripheral light rays entering the eye at visual angles in a range of about 50 degrees to about 80 degrees. By way of example, in many embodiments the central portion of each surface is characterized by a radial distance from an optical axis of the optic (e.g., an axis about which the central portion is rotationally symmetric) in a range of about 2 mm to about 3.5 mm while the respective peripheral extension is characterized by a maximum radial extension from that axis in a range of about 2.5 mm to about 4.5 mm.

In many embodiments, the optic is foldable to facilitate its insertion into the eye, and the peripheral extension is rotationally asymmetric about the optical axis so as to ensure that the IOL can be inserted in a folded state into the eye through a small incision. In many embodiments, the peripheral extension can be in the form of a crescent-shaped section that partially surrounds the central portion. In some such embodiments, each of the anterior and posterior surfaces can be characterized by two orthogonal meridians one of which exhibits a radial extension relative to the optical axis greater than about 3.5 mm and other exhibits a smaller radial extension (e.g., less than about 3.1 mm).

In some embodiments in which the peripheral extension of the IOL has a focusing function, at least one of the anterior or posterior surfaces exhibits an asphericity to ameliorate, and preferably prevent, spherical aberration effects that might arise as a result of focusing of the rays entering at large visual angles into the eye. By way of example, such an asphericity can be characterized by a conic constant in a range of about −10 to about −100, and preferably in a range of about −15 to about −25. In other embodiments, the IOL can include one or more toric surfaces.

In another aspect, in the above IOL, a diffractive structure can be disposed on at least one of the surfaces of the optic such that the IOL would provide a far-focus as well as a near-focus power. In some cases, the diffractive structure includes a plurality of diffractive zones that are separated from one another by steps that exhibit decreasing heights as a function of increasing radial distance from the optical axis so as to change the balance of energy diverted to the near and far foci based on the pupil size.

In another aspect, an IOL is disclosed that includes an optic disposed about an optical axis, where the optic provides an optical power for generating an image of a field of view on the retina of a patient's eye in which the IOL is implanted. The IOL further includes an optical flange that at least partially surrounds the optic, where the flange is adapted to inhibit dysphotopsia, e.g., by inhibiting the formation of a secondary image by peripheral light rays entering the eye at large visual angles or by directing some light rays into a shadow region between the image formed by the IOL and such a secondary image.

In a related aspect, in the above IOL, the optic has a maximum radial extension in a range of about 2 mm to about 3.5 mm relative to the optical axis and the optical flange has a maximum radial extension in a range of about 2.5 mm to about 4.5 mm from that axis.

In other aspects, the optical flange can include at least one surface that is textured (e.g., it is characterized by physical surface undulations with amplitudes in a range of about 0.2 microns to about 2 microns) so as to scatter peripheral light rays incident thereon in order to inhibit those rays from forming a secondary image, or to redirect at least some of the light rays into the shadow region. Alternatively or in addition, the optical flange can be opaque or translucent to visible light. In some cases, the optical flange can include a diffractive structure or a Fresnel lens.

Further understanding of various aspects of the invention can be obtained by reference to the following detailed description in conjunction with the associated drawings, which are described briefly below.

### BRIEF DESCRIPTION OF THE DRAWINGS

FIG. 1A is a schematic top view of an IOL according to one embodiment of the invention,

FIG. 1B is a schematic side view of the IOL depicted in FIG. 1A,

FIG. 1C is a schematic perspective view of the IOL depicted in FIG. 1A,

FIG. 1D schematically depicts the anterior surface of the IOL of FIG. 1A, indicating that the surface can be characterized by two orthogonal meridians having different radial extensions from the IOL's optical axis,

FIG. 1E schematically depicts an IOL according to another embodiment whose optic includes a central portion that extends to an asymmetric extension that is slanted relative to the central portion,

FIG. 2A and 2B schematically depict that the IOL of FIG. 1A can be folded to facilitate its insertion in the eye

FIG. 3A schematically shows a conventional IOL implanted in a patient's eye, illustrating schematically the formation of a secondary image by peripheral light rays that enter the eye at large visual angles and miss the IOL,

FIG. 3B schematically shows an IOL according to one embodiment of the invention implanted in a patient's eye, illustrating schematically that the IOL's peripheral portion inhibits formation of a secondary image by peripheral light rays entering the eye at large visual angles.

FIG. 4A is a schematic top view of an IOL according to another embodiment of the invention,

FIG. 4B is a schematic side view of the IOL shown in FIG. 4A,

FIG. 4C is a schematic perspective view of the IOL shown in FIGS. 4A and 4B,

FIG. 5A schematically depicts the IOL of FIGS. 4A-4C implanted in a patient's eye, illustrating that the IOL inhibits dysphotopsia by preventing peripheral light rays entering the eye at large visual angles from forming a secondary image,

FIG. 5B schematically depicts an IOL according to an embodiment of the invention, which inhibits dysphotopsia once implanted in a patient's eye by causing scattering of some light rays into a shadow region between an image formed by the IOL and a second peripheral image formed by light rays entering the eye that miss the IOL,

FIG. 6A is a schematic side view of an IOL in accordance with another embodiment of the invention,

FIG. 6B is a schematic perspective view of the IOL of FIG. 6A,

FIG. 6C is a schematic side view of an IOL according to another embodiment having a diffractive structure on a peripheral extension thereof,

FIG. 6D is a schematic side view of an IOL according to another embodiment having a Fresnel lens on a peripheral extension thereof,

FIG. 7 is a schematic side view of an IOL in accordance with another embodiment of the invention,

FIG. 8A is a schematic side view of an IOL according to another embodiment of the invention having a central optic and a peripheral flange,

FIG. 8B is a schematic anterior view of the IOL of FIG. 8A,

FIG. 9 is a schematic side view of an IOL in accordance with another embodiment of the invention having a central optic and a peripheral flanges, where a surface of the flange is textured for scattering of the light rays incident thereon,

FIG. 10 is a schematic side view of an IOL in accordance with another embodiment of the invention having a central optic and a peripheral flange, where the surfaces of the flange are opaque to visible light,

FIG. 11 is a schematic side view of an IOL in accordance with another embodiment of the invention having a central optic and a peripheral flange, where the peripheral flange provides focusing of light rays incident thereon,

FIG. 12A is a schematic side view of an IOL according to another embodiment having a diffractive structure on a peripheral flange thereof,

FIG. 12B is a schematic side view of an IOL according to another embodiment having a Fresnel lens on a peripheral flange thereof,

FIG. 12C schematically depicts an IOL according to another embodiment that includes a central optic that is partially surrounded by a flange, which is slanted relative to the optic,

FIG. 13A is a schematic side view of an IOL in accordance with another embodiment of the invention having a diffractive structure on an anterior surface thereof, and

FIG. 13B is a schematic anterior view of the IOL of FIG. 13A.

### DETAILED DESCRIPTION

The present invention generally provides intraocular lenses (IOLs) that ameliorate, and preferably prevent, the perception of dark shadows that some IOL patients report. As noted above, such an effect is known generally in the art as dysphotopsia. As discussed in more detail below, in many embodiments, the IOLs of the invention include larger optics with asymmetric profiles that can be characterized as having a central portion that extends to a peripheral extension. In many cases, the peripheral extension can receive peripheral light rays entering the eye at large visual angles and can capture or redirect those rays to inhibit the perception of peripheral visual artifacts (e.g., shadows) by the IOL user. In some cases, the surfaces of the IOL's optic are extended in certain directions (typically in the nasal direction) to provide the peripheral extension. In other cases, the peripheral extension is in the form of a separate flange that partially surrounds a central optic. The term “intraocular lens” and its abbreviation “IOL” are used herein interchangeably to describe lenses that are implanted into the interior of the eye to either replace the eye's natural lens or to otherwise augment vision regardless of whether or not the natural lens is removed.

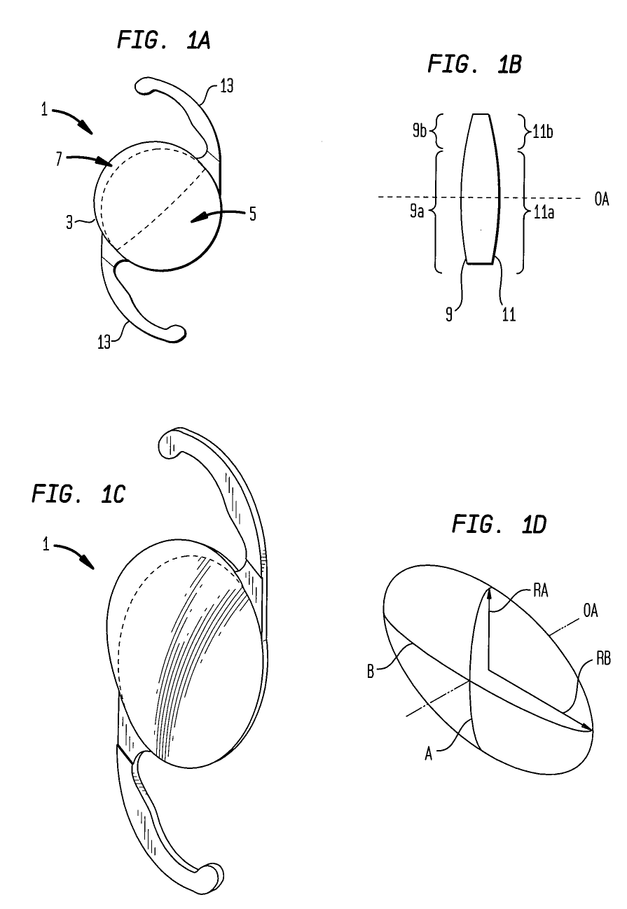

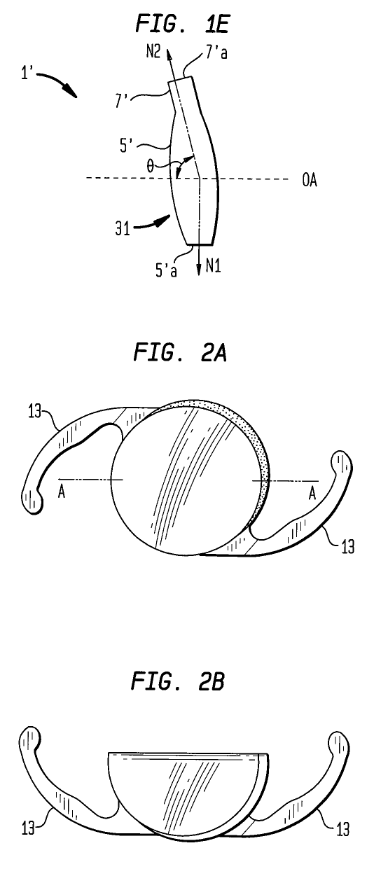

FIGS. 1A, 1B and 1C schematically show an intraocular lens (IOL) 1 in accordance with one embodiment of the invention that includes an optic 3 having an asymmetric profile, where the optic has larger radial sizes along certain directions than others. More specifically, in this embodiment, the optic is extended in the nasal direction (i.e., the direction closer to the nose once the IOL is implanted in the eye) so as to receive peripheral light rays entering the eye at large visual angles. The term “large visual angles,” as used herein, refers to angles relative to the visual axis of the eye that are greater than about 50 degrees, and are typically in a range of about 50 degrees to about 80 degrees relative to the eye's visual axis. The optic 3 can be characterized as having a central portion 5 that extends to a peripheral extension 7. In this embodiment, the peripheral extension is in the form of a single crescent-shaped section that extends partially about the central portion. While the central portion is rotationally symmetric about an axis OA (herein also referred to as optical axis OA), the peripheral extension is rotationally asymmetric about that axis. Hence, the peripheral extension causes the optic 3 as a whole to be rotationally asymmetric about the axis OA. In fact, in this embodiment, the optic 3 lacks not only continuous rotational symmetry about axis OA but it also lacks discrete rotational symmetry about that axis (e.g., for a rotation by 180 degrees).

More specifically, with reference to FIG. 1B, the optic 3 includes an anterior surface 9 and a posterior surface 11. The surfaces 9 and 11 can be characterized as having, respectively, central portions 9a and 11a that extend to respective peripheral surface extensions 9b and 11b. While the central portions 9a and 11a are rotationally symmetric about the axis OA, the peripheral extensions 9b and 11b are rotationally asymmetric about that axis. In particular, in this embodiment, each of the peripheral extensions 9b and 11b only partially surrounds the respective central portions 9a and 11a. Hence, the optic exhibits an asymmetric profile. In this embodiment, each of the anterior and the posterior surfaces can be characterized by two orthogonal meridians, one of which corresponds to a maximum radial extension of that surface and the other to a minimum radial extension. By way of example, FIG. 1D shows such two meridians A and B for the anterior surface 9. While meridian A is characterized by a radial distance RA relative to the optical axis in a range of about 2 millimeters (mm) to about 3.5 mm, meridian B is characterized by a respective radial distance RB that is in a range of about 2.5 mm to about 4.5 mm.

The optic 3 is preferably formed of a biocompatible material, such as soft acrylic, silicone, hydrogel, or other biocompatible polymeric materials having a requisite index of refraction for a particular application. For example, in some embodiments, the optic can be formed of a cross-linked copolymer of 2-phenylethyl acrylate and 2-phenylethyl methacrylate, which is commonly known as Acrysof®. The IOL 1 also includes a plurality of fixation members (haptics) 13 that facilitate its placement in the eye. Similar to the optic 3, the haptics 13 can also be formed of a suitable biocompatible material, such as polymethylmethacrylate (PMMA). While in some embodiments, the haptics can be formed integrally with the optic, in other embodiments (commonly referred to as multipiece IOLs), the haptics are formed separately and attached to the optic in a manner known in the art. In the latter case, the material from which the haptics are formed can be the same as, or different from, the material forming the optic. It should be appreciated that various haptic designs for maintaining lens stability and centration are known in the art, including, for example, C-loops, J-loops, and plate-shaped haptic designs. The present invention is readily employed with any of these haptic designs.

Referring again to FIG. 1A, the orientation of the peripheral extension 7 relative to the haptics 13 is provided as one example, and it can be different in other embodiments than that shown in the figure. By way of example, in some implementations, a portion of the extension 7 can form a connecting junction between one of the haptics and the central portion 5.

Further in some implementations, the peripheral extension of the IOL's optic can be slanted anteriorly or posteriorly relative to its central portion. By way of example, FIG. 1E, an IOL 1′ can include an optic 3′ having a central portion 5′ that extends to a peripheral extension 7′, which is slanted relative to the central portion. More particularly, a normal N1 to an edge surface 5′a of the central portion is substantially orthogonal to an optical axis OA whereas a normal N2 to a surface 7′a of the extension forms an angle θ relative to the optical axis. Further, in some implementations of this or other embodiments, the extension's thickness can be less than the minimum (or the average) thickness of the central portion (e.g., by a factor of about 5).

With reference to FIGS. 2A and 2B, the IOL 1 is foldable, e.g., about an axis A, to facilitate its implantation in the eye. As the peripheral extension 7 is asymmetric about the optic, it allows folding of the IOL such that it can be readily inserted through a small incision into the eye. By way of illustration, FIG. 2B schematically shows the IOL 1 in a folded state, which can be readily inserted through an incision that can accommodate its transverse size into the eye.

More particularly, during cataract surgery, a clouded natural lens can be removed and replaced with the IOL 1. By way of example, an incision can be made in the cornea, e.g., via a diamond blade, to allow other instruments to enter the eye. Subsequently, the anterior lens capsule can be accessed via that incision to be cut in a circular fashion and removed from the eye. A probe can then be inserted through the corneal incision to break up the natural lens via ultrasound, and the lens fragments can be aspirated. An injector can be employed to place the IOL in a folded state in the original lens capsule. Upon insertion, the IOL can unfold and its haptics can anchor it within the capsular bag.

In some cases, the IOL is implanted into the eye by utilizing an injector system rather than employing forceps insertion. For example, an injection handpiece having a nozzle adapted for insertion through a small incision into the eye can be used. The IOL can be pushed through the nozzle bore to be delivered to the capsular bag in a folded, twisted, or otherwise compressed state. The use of such an injector system can be advantageous as it allows implanting the IOL through a small incision into the eye, and further minimizes the handling of the IOL by the medical professional. By way of example, U.S. Pat. No. 7,156,854 entitled “Lens Delivery System,” which is herein incorporated by reference, discloses an IOL injector system. The IOLs according to the embodiments of the invention, such as the IOL 1, are preferably designed to inhibit dysphotopsia while ensuring that their shapes and sizes allow them to be inserted into the eye via injector systems through small incisions.

Once implanted in a patient's eye, the IOL can form an image of a field of view with its peripheral extension receiving peripheral light rays entering the eye at large visual angles and directing those rays towards the image, thereby inhibiting formation of a secondary image that could lead to perception of dark shadows (that is, the peripheral extension inhibits dysphotopsia). In this embodiment, the peripheral extension 7 is adapted to be positioned, upon implantation of the IOL in the eye, on the nasal side of the eye such that the temporal peripheral light rays would be incident thereon—the nose, eyebrows and cheeks typically block the entry of peripheral light rays from other directions into the eye.

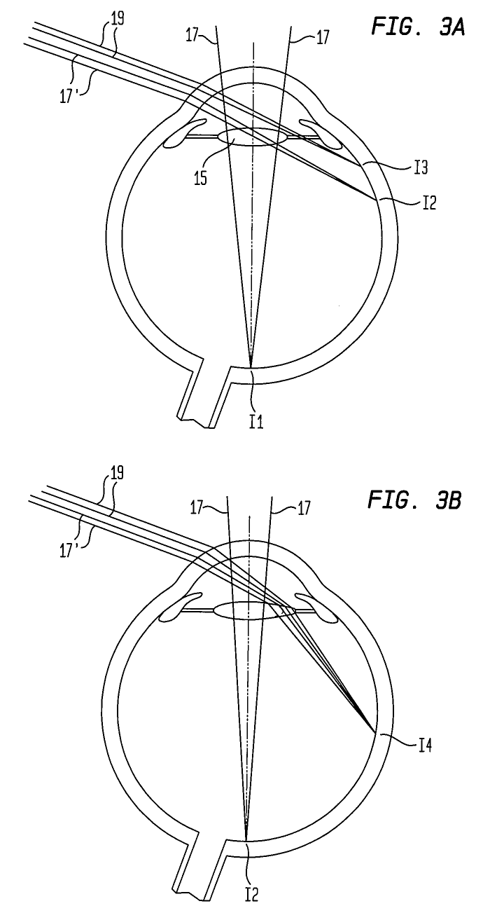

To further illustrate the role of the peripheral extension in inhibiting dysphotosia, FIG. 3A shows a conventional IOL 15 implanted in the eye and FIG. 3B shows the above IOL 1 implanted in the eye. With reference to FIG. 3A, the conventional IOL 15 can form an image of a field of view by focusing a plurality of light rays (such as central rays 17 and peripheral rays 17′) entering the eye onto the retina. Such an image can be characterized as having a central (axial) portion I1 and a temporal peripheral portion I2. However, a plurality of peripheral light rays (such as rays 19) that enter the eye at large visual angles are refracted by the cornea but miss the IOL 15. As such, these peripheral rays reach the retina at a location separated from the image generated by the IOL to form in many cases a secondary image I2. The formation of such a secondary image can result in the perception of a shadow-like phenomenon by the patient between those images.

In contrast, as shown schematically in FIG. 3B, the peripheral extension 7 of the IOL 1 receives temporal peripheral light rays entering the eye at large visual angles and focuses those rays onto the retina so as to form a single image of the field of view, in which the rays focused by the extension 7 are directed to the nasal peripheral portion of the image (I4). In other words, the peripheral extension 7 augments the temporal peripheral vision without generating a displaced second image, which could lead to perception of shadows. Instead, the IOL's central portion and its peripheral extension function as a single focusing unit to form a single image of a field of view by focusing the light rays entering the eye over a range of visual angles, including those entering the eye from the temple side at large visual angles. In this manner, the IOL 1 inhibits dysphotopsia.

In some embodiments, at least one of the anterior or posterior surfaces of the IOL 1 exhibits an asphericity designed to ameliorate, and preferably prevent, spherical aberration effects that may arise from focusing of the peripheral light rays by the peripheral extension 7. By way of example, at least one of the surfaces can exhibit an asphericity characterized by a conic constant in a range of about −10 to about −100, or in a range of about −15 to about −25. Further, in some cases one or more surfaces of the IOL can have a toric profile (i.e. a profile characterized by two different optical powers along two orthogonal surface directions). Additional teachings regarding the use of aspheric and/or toric surfaces in IOLs, such as various embodiments discussed herein, can be found in U.S. patent application Ser. No. 11/000,728 entitled “Contrast-Enhancing Aspheric Intraocular Lens,” filed on Dec. 1, 2004 and published as Publication No. 2006/0116763, which is herein incorporated by reference in its entirety.

In other embodiments, the IOL of the invention can include a peripheral extension characterized by at least one textured, opaque, and/or translucent surface. Such peripheral extensions can inhibit the formation of a second peripheral image or redirect light rays into the shadow region to inhibit perception of a dark shadow by the IOL user.

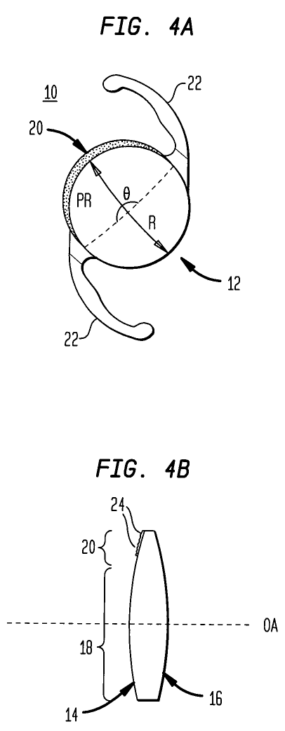

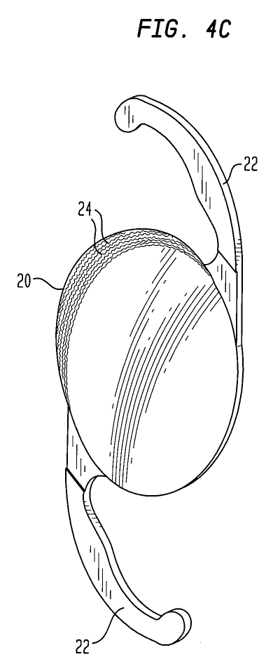

By way of example, FIGS. 4A, 4B and 4C schematically depict different views of an exemplary intraocular lens (IOL) 10 according to one such embodiment of the invention that includes an optic 12 formed of an anterior surface 14 and a posterior surface 16 disposed about an optical axis OA. The optic 12 comprises a central portion 18 that extends to a peripheral extension 20 that partially surrounds it. As discussed in more detail below, once the IOL is implanted in a patient's eye, the peripheral light rays entering the eye at large visual angles from the temporal side are incident on the peripheral extension, which inhibits those rays from forming a second peripheral image so as to ameliorate, and preferably prevent, the perception of dark shadow-like effects that might otherwise occur in a region between the image and a putative secondary image.

More particularly, in this embodiment, the central portion exhibits a substantially circular cross section, with the optical axis connecting the centers of the central portions of the anterior and posterior surfaces, as shown schematically in FIG. 1B. While the central portion is rotationally symmetric about the axis OA, the peripheral extension is rotationally asymmetric about that axis. Rather than completely surrounding the central portion 18, the peripheral extension 20 spans about an arc subtended by an angle θ, which in this embodiment is about 160 degrees, though in other embodiments it can be in a range of about 30 degrees to about 80 degrees. In this embodiment, the central portion of the anterior or the posterior surface can be characterized by a radial distance R from the optical axis OA, which can be equal or greater than about 2 mm. By way of example, in many cases, the radius of the central portion is in a range of about 2 mm to about 3.5 mm. The peripheral extension of each of the anterior and posterior surfaces can, in turn, exhibit a maximum distance (PR) from the optical axis that is preferably equal or greater than about 2.5 mm, e.g., in a range of about 2.5 mm to about 4.5 mm.

Similar to the previous embodiment, the optic 12 is preferably formed of a biocompatible material, such as those discussed above. The IOL 10 also includes a plurality of fixation members (haptics) 22 that facilitate its placement in the eye. Similar to the optic 12, the haptics 22 can also be formed of a suitable biocompatible material, such as PMMA. While in some embodiments, the haptics can be formed integrally with the optic, in other embodiments, the haptics are formed separately and attached to the optic in a manner known in the art. Further, similar to the previous embodiment, the IOL 10 is foldable so as to facilitate its insertion in the eye.

In this embodiment, the peripheral extension of the optic is textured in order to cause scattering of the peripheral light rays entering the eye at large visual angles so as to ensure that those rays would not form a discernible second peripheral image on the retina whose separation from a primary image formed by the optic's central portion would lead to the perception of dark shadows. More specifically, as shown schematically in FIGS. 4B and 4C, the peripheral extension of the anterior surface 14 exhibits surface undulations 24 (that is, the anterior peripheral surface extension is textured) with amplitudes typically of the order of wavelengths of the visible light. In this embodiment, the peripheral extension of the posterior surface 16 is not textured (the posterior surface has a smooth surface profile) so as to minimize the potential of posterior capsular opacification (PCO)—though in other embodiments both the anterior and posterior peripheral extension or only the posterior peripheral extension can be textured.

In many embodiments, the surface undulations have amplitudes that create an optical path distance effect of the order of visible light wavelengths. For example, in some embodiments, the physical surface amplitudes can range from about 0.2 microns to about 2 microns. As discussed in more detail below, upon implantation of the IOL in a patient's eye to replace a clouded natural lens, the surface undulations can cause scattering of peripheral light rays incident thereon, and hence inhibit formation of an image by those rays.

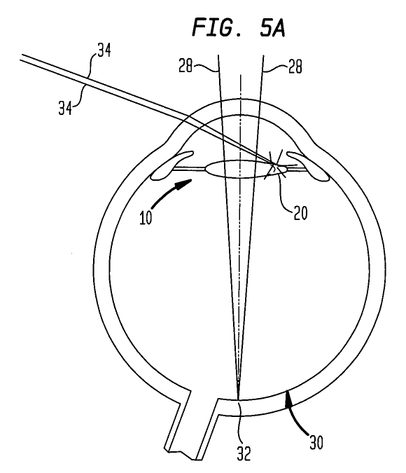

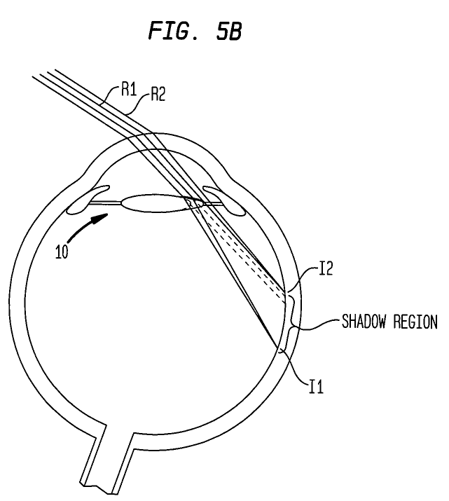

In this embodiment, once the IOL is implanted in the eye, its peripheral extension 20 is positioned on the nasal side of the eye in order to receive peripheral rays entering the eye from the temporal side, as discussed below. More specifically, with reference to FIGS. 5A, the IOL can form an image of a field of view by focusing light rays, such as rays 28, incident thereon onto the retina, with the peripheral extension receiving temporal peripheral rays (such as rays 34) that enter the eye at large visual angles. As the peripheral extension of the IOL 10 includes a textured surface, the incident peripheral rays are scattered, as shown schematically in FIG. 5A, rather than being focused onto the retina. Although some of the scattered rays might reach the retina, they do not form a strong secondary image that would result in perception of dark shadows.

In other words, the IOL's peripheral extension effectively increases the IOL's size on the nasal side, which moves the IOL's edge further into the far temple visual field. This allows the IOL to capture the temporal peripheral rays 42 and substantially inhibit, and preferably prevent, them, via scattering, from forming a secondary image.

In some embodiments, the IOL's textured extension, rather than inhibiting formation of a second peripheral image, scatters some of the light rays incident thereon into a shadow region between a primary image formed by the IOL and a second peripheral image generated by rays that miss the IOL and are focused only by the cornea onto the retina. By way of example, FIG. 5B schematically shows that some of the peripheral light rays entering the eye at very large visual angles (such an exemplary rays R1 and R2) might miss the IOL 10 to form, via refraction by the cornea, a second peripheral image I2. As noted above, this can in fact be beneficial as the secondary image expands the peripheral visual field. However, to inhibit perception of a dark shadow between this image and the peripheral edge (I1) of an image formed by the IOL, the IOL's textured extension scatters some of the light incident thereon to the shadow region. In some cases, the textured surface scatters only a portion of peripheral light rays incident thereon (e.g., less than about 40%) and focuses the other rays onto the retina to enhance the IOL user's peripheral vision.

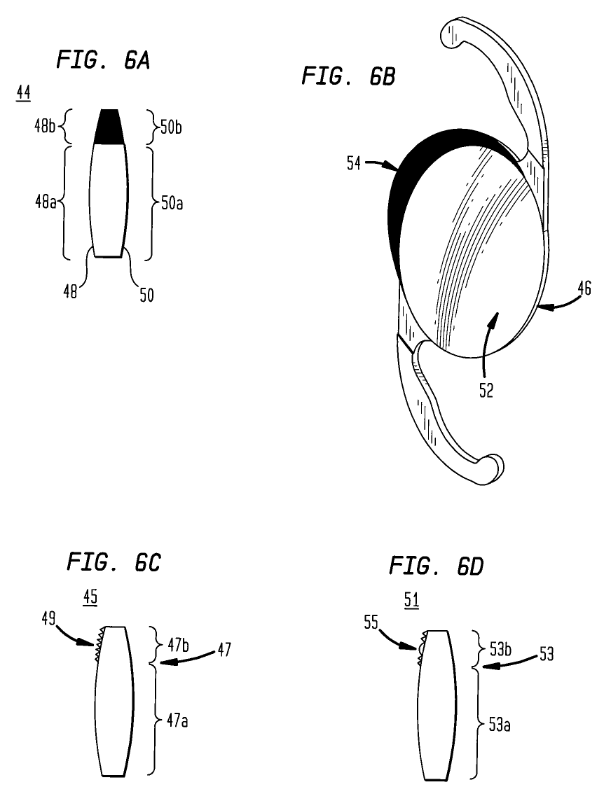

Although in some of the above embodiments, the peripheral extension of the IOL 10 inhibits formation of a secondary peripheral image by scattering the peripheral rays incident thereon, in some other embodiments, the peripheral extension can be opaque to visible radiation so as to significantly reduce, and in some cases eliminate, the intensity of peripheral rays that pass through it to reach the retina. While in some cases the opaque peripheral extension prevents such peripheral rays, e.g., via absorption, from reaching the retina, in other cases it can redirect such rays to the retina but at a reduced intensity (it can absorb some of the rays, but allow the passage of others). By way of example, FIGS. 6A and 6B schematically show an IOL 44 in accordance with such an embodiment that includes an optic 46 composed of an anterior surface 48, having a central portion 48a and a peripheral surface extension 48b, and a posterior surface 50, having a central portion 50a and a peripheral surface extension 50b. Similar to the previous embodiments, the optic 46 includes a central portion 52 that extends to a peripheral extension 54, where the central portion can form an image of a visual field of view when the IOL is implanted in a patient's eye.

The peripheral extension is, however, substantially opaque to visible radiation so as to inhibit the light rays entering the eye at large visual angles from reaching the retina, thus preventing the formation of a secondary image. The term “opaque to visible radiation,” as used herein, refers to an opacity that would result in a reduction in the intensity of the visible radiation, e.g., radiation with wavelengths in a range about 380 nm to about 780 nm, by more than about 25%, or by more than about 40%, or by more than about 90%, or by more than about 95%, or by 100%. By way of example, in many embodiments, the intensity of the incident light passing through the opaque peripheral extension is reduced by a factor greater than about 25%, and more preferably greater than about 50%.

The opaque portion of the optic can be formed by a variety of techniques, e.g., by impregnating the polymeric material with one or more suitable dye(s). Some examples of dyes that can be in used are provided in U.S. Pat. No. 5,528,322 (entitled “Polymerizable Yellow Dyes And Their Use In Ophthalmic Lenses”), U.S. Pat. No. 5,470,932 (entitled “Polymerizable Yellow Dyes And Their Use In Ophthalmic Lenses”), U.S. Pat. No. 5,543,504 (entitled “Polymerizable Yellow Dyes And Their Use In Ophthalmic Lenses), and U.S. Pat. No. 5,662,707 (entitled “Polymerizable Yellow Dyes And Their Use In Ophthalmic Lenses), all of which are herein incorporated by reference. Further, while in this embodiment the entire peripheral extension is opaque, in other embodiments such opacity can be imparted to only portions of the peripheral extension, e.g., portions in proximity of the extension's anterior and/or posterior surfaces.

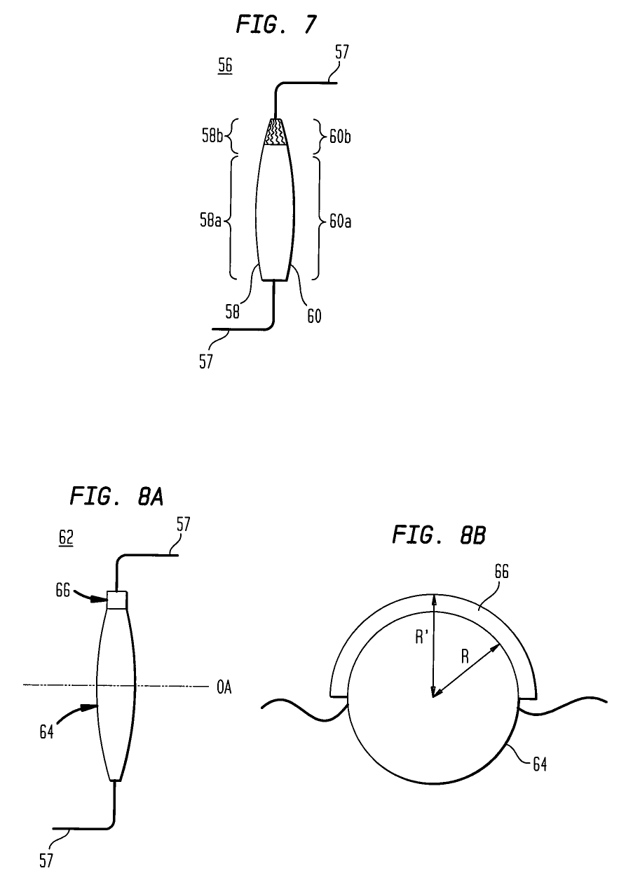

In other embodiments, the peripheral extension of the optic can be translucent so as to inhibit the peripheral light rays that enter the eye at large visual angles from generating a secondary image, or to redirect some light into a shadow region between such a secondary image and an image formed by the IOL. By way of example, FIG. 7 schematically depicts an IOL 56 having an anterior surface 58 and a posterior surface 60. Similar to the previous embodiments, each of the anterior and the posterior surfaces includes a central portion (depicted as portions 58a and 60a of the anterior and the posterior surfaces, respectively) that extends to a peripheral extension (depicted as portions 58b and 60b of the anterior and posterior surface, respectively). In this embodiment, the peripheral extension is translucent. As such, it allows the peripheral light rays to pass therethrough, but diffusely. This can prevent formation of a secondary image, or redirect at least some of the light rays into a shadow region between such a secondary peripheral image and a primary image formed by the lens, thereby preventing or at least ameliorating dysphotopsia. In some cases, the peripheral extension can be made translucent by creating surface undulations (roughness) with amplitudes in a range of about 0.2 microns to about 2 microns, and preferably in a range of about 0.2 microns to about 0.4 microns. Further, the IOL 56 includes haptics 57 that facilitate its placement in a patient's eye.

In some cases, a diffractive structure or a Fresnel lens can be disposed on a surface of the peripheral extension to direct light incident thereon onto a reduced intensity retinal region between an image formed by the central portion of the optic and a second peripheral image formed by light rays entering the eye that miss the optic. For example, FIG. 6C depicts an IOL 45 having an optic 47, which includes a central portion 47a extending to an asymmetric peripheral extension 47b with a diffractive structure 49 that is disposed on an anterior surface the extension. The diffractive structure is adapted to direct at least some of the light incident thereon to the shadow region. In some implementations, the diffractive structure can provide a focusing power that is less than that of the central portion of the optic (e.g., by a factor in a range of about 25% to about 75%). By way of example, the diffractive structure can be formed of a plurality of diffractive zones, each of which is separated from an adjacent zone by a step. The step heights can be uniform or non-uniform. In some exemplary implementations, the step heights are uniform and can be represented by the following relation:

$$Step height = \frac{λ}{a ( {n}_{2} - {n}_{1} )} \qquad Eq . ( 1 )$$

wherein,
λ denotes a design wavelength (e.g., 550 nm);
a denotes a parameter that can be adjusted to control diffraction efficiency associated with various orders, e.g., a can be selected to be 1,
n₂ denotes the index of refraction of the optic, and
n₁ denotes the refractive index of a medium in which the lens is placed.

With reference to FIG. 6D, in another embodiment, an IOL 51 can include an optic 53 having a central portion 53a that extends to an asymmetric peripheral extension 53b. A Fresnel lens 55 is disposed on an anterior surface of the peripheral extension and is adapted to direct at least some of the light incident thereon to the shadow region. In some cases, the Fresnel lens provides a focusing power less than that of the optic's central portion (e.g., by a factor in a range of about 25% to about 75%).

In many embodiments, such as those discussed above, the peripheral extension spans partially around the central portion of the optic. The extent by which the peripheral extension spans around the central portion can vary from one embodiment to another. In many cases the angular span of the peripheral extension is selected based on the following considerations: (1) ensuring that the peripheral extension would receive sufficient number of peripheral rays to inhibit perception of dark shadows and (2) ensuring that the increase in the size of the IOL would not hinder its insertion in the eye. By way of example, in many embodiments an angle θ corresponding to an arc spanned by the peripheral extension can be in a range of about 30 degrees to about 80 degrees.

In the above embodiments, the peripheral extension of each surface of the optic is integrally formed with its central portion. In some other embodiments, the IOL can include a central optic and an asymmetric separate flange that is coupled to the optic's periphery (e.g., it can abutt against the optic) by employing known techniques in the art.

By way of example, FIGS. 8A and 8B schematically depict an intraocular lens (IOL) 62 according to such an embodiment that includes a central optic 64 and an optical flange 66 that partially surrounds the central optic. Both the central optic and the peripheral flange are formed of suitable biocompatible materials, such as those discussed above. In this exemplary embodiment, the central optic is rotationally symmetric about an optical axis OA, while the peripheral optical flange 66 is rotationally asymmetric about that axis.

In many embodiments, the peripheral flange is adapted to receive, once the IOL is implanted in the eye, at least some of the light rays entering the eye at large visual angles in a range of about 50 to about 80 degrees. In some embodiments, the central optic has a radius R relative to the optical axis in a range of about 2 mm to about 3.5 mm, and the peripheral flange has a maximum radial distance (R′) from the optical axis in a range of about 2.5 mm to about 4.5 mm.

In this embodiment, once the IOL is implanted in the eye, the central optic 64 focuses the light rays incident thereon onto the retina so as to form an image of a field of view, while the optical flange 66 is adapted to be on the nasal side of the eye so as to receive at least a portion of the temporal peripheral rays. As discussed in more detail below, the flange 66 can inhibit such peripheral rays from forming a secondary image on the retina displaced from the image formed by the central optic that would lead to negative dysphotopsia, or redirect light into a shadow region between the peripheral edge of an image formed by central optic and secondary peripheral image formed by rays that miss the IOL. For example, the optical flange can function as a focusing element to redirect the peripheral light rays incident thereon to the retina so as to form, together with the central optic, a single image of a field of view. Alternatively, the optical flange can include one or more textured, opaque and/or translucent surface(s) that would inhibit the peripheral light rays from forming a secondary image, or redirect those rays into the shadow region.

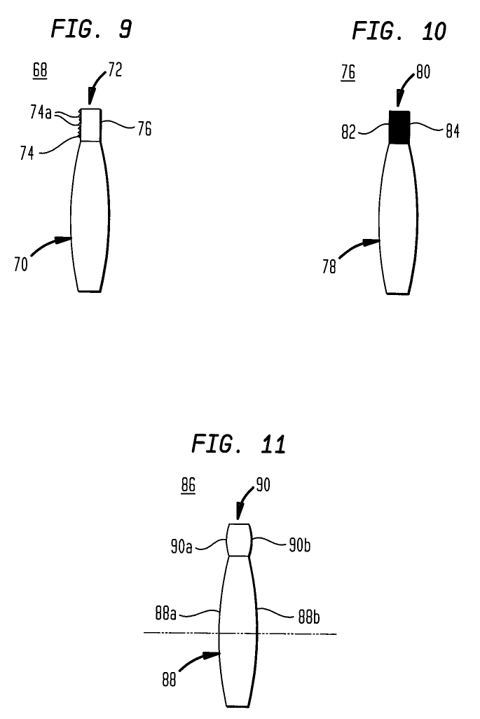

By way of example, in some embodiments, at least one surface of the optical flange is textured to cause sufficient scattering of the peripheral rays so as to inhibit those rays from forming a secondary image on the retina. For example, FIG. 9 shows an IOL 68 having a central optic 70 and a peripheral optical flange 72 that spans partially about the central optic. The optical flange comprises an anterior surface 74 and a posterior surface 76, both of which are substantially flat—though curved surfaces can also be employed. The anterior surface 74 of the flange is textured so as to cause scattering of the peripheral light rays incident thereon. More specifically, the textured surface 74 is characterized by a plurality of surface undulations 74a, which typically have physical surface amplitudes in a range of about 0.2 microns to about 2 microns. In some cases, the scattering caused by the textured surface distributes at least about 40%, or at least about 90%, or at least 95%, of the incident light randomly over a plurality of directions. Although in other embodiments the posterior surface of the flange 72, or both the anterior and posterior flange surfaces, can be textured, it is preferable that only the anterior surface be textured so as to reduce the potential risk of PCO.

In this exemplary embodiment, upon implantation of the IOL in a patient's eye, the peripheral optical flange 72 is positioned on the nasal side of the IOL such that the peripheral light rays entering the eye at large visual angles from the temporal side would be incident thereon. The textured anterior surface of the flange causes scattering of such peripheral rays, thereby inhibiting the formation of a secondary image by those rays. Alternatively, in some embodiments, the textured flange surface can scatter some light rays incident thereon into a shadow region between a secondary peripheral image, formed by peripheral rays that might miss the IOL, and a primary image formed by the IOL.

In other embodiments, the optical flange is opaque to visible radiation so as to substantially inhibit (via reduction in intensity), and or in some cases prevent, the peripheral rays from reaching the retina. By way of example, FIG. 10 schematically depicts an intraocular lens (IOL) 76 having a central optic 78 and a peripheral optical flange 80. The optical flange includes an anterior surface 82 and a posterior surface 84. The optical flange is opaque to visible radiation so as to inhibit the peripheral light rays from striking the retina at a location sufficiently removed from a primary image formed by the central optic to be perceived as a second peripheral image. In this manner, the peripheral flange can ameliorate, and preferably prevent, the perception of a dark shadow by a patient in whose eye the IOL is implanted.

With continued reference to FIG. 10, in some embodiments, the opacity of the optical flange is such it reduces the intensity of peripheral light rays by more than about 25%, or by more than about 40%, or by more than about 90%. While in this exemplary embodiment, the entire flange is opaque, in other embodiments, only certain portions thereof can be opaque, e.g., portions in proximity of the flange's anterior and/or posterior surfaces. Further, in some embodiments, at least one surface of the flange, e.g., its anterior surface, can be textured while the flange is also opaque and/or translucent. Further, while in some embodiments in which the flange is opaque the degree of opacity across the flange is substantially uniform, in other embodiments the flange can exhibit a graded opacity.

In yet other embodiments, the peripheral flange can include one or more curved surfaces adapted to direct the peripheral rays entering the eye at large visual angles towards the periphery of an image formed by the central optic on the patient's retina to enhance the IOL user's peripheral vision while inhibiting dysphotopsia. By way of example, FIG. 11 schematically depicts an IOL 86 having a central optic 88 to which an optical flange 90 is coupled. The central optic 88 is in the form of a biconvex lens comprising an anterior surface 88a and a posterior surface 88b, though other shapes such as plano-convex or plano-concave are also possible. The curvatures of the anterior and the posterior surfaces are selected such that the central optic would provide a desired optical power, e.g., in a range of about −15 to about +40 D, for generating an image of a field of view. Though not shown, the IOL 86 can include haptics for secure implantation in the eye.

With continued reference to FIG. 11, the peripheral flange is also formed of an anterior surface 90a and a posterior surface 90b, both of which are curved. In many embodiments, the curvatures of those surfaces are such that the flange would provide an optical power that is substantially the same as that of the central optic 88. In such embodiments, the flange would focus the peripheral light rays incident thereon onto the retina such that they would form, together with the rays focused by the central optic, a single image of a field of view.

In some other embodiments, the optical power provided by the flange is slightly less than that of the central optic. For example, the optical power of the flange can differ from that of the central optic by a factor in a range of about 25% to about 75%. By way of example, in some embodiments, the optical power of the flange is less than by about 50% than that of the optic.

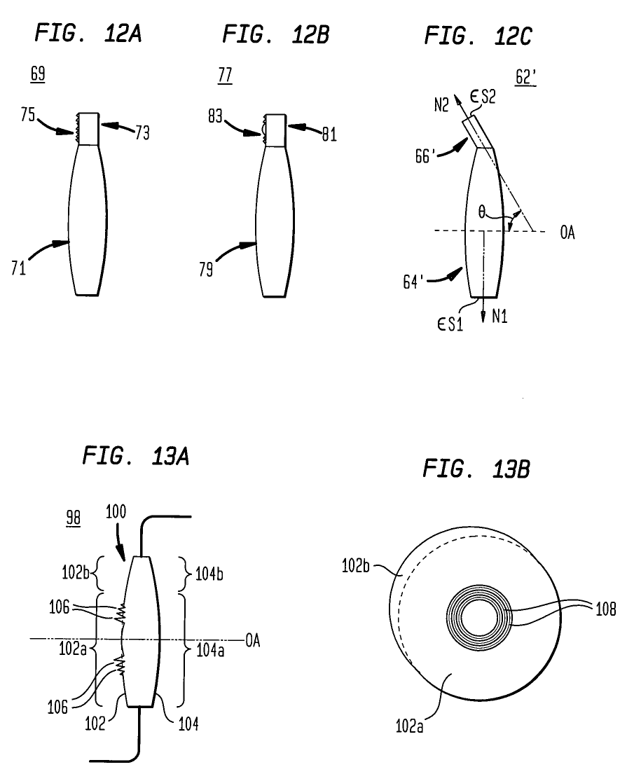

In some embodiments, a diffractive structure or a Fresnel lens can be disposed on at least a surface of the peripheral flange to direct light incident thereon to the reduced intensity retinal region. By way of example, FIG. 12A schematically depicts an IOL 69 having an optic 71 that is partially surrounded by a peripheral flange 73. A diffractive structure 75, e.g., one similar to that described above in connection with FIG. 6C, is disposed on an anterior surface of the flange and is adapted to direct at least some of the light incident thereon to the shadow region. By way of another example, FIG. 12B depicts an IOL 77 having an optic 79 that is surrounded partially by a peripheral flange 81, where the flange includes a Fresnel lens 83 on an anterior surface thereof. Again, the Fresnel lens is adapted to direct some of the light rays incident thereon to the shadow region. In some implementation of the IOLs 69 and 77, the focusing power provided by the diffractive structure or the Fresnel lens is less than that of the central portion of the IOL's optic.

Further in some implementations, the IOL's peripheral flange can be slanted anteriorly or posteriorly relative to its central optic. By way of example, with reference to FIG. 12C, an IOL 62′ can include an optic 64′ that is partially surrounded by a peripheral flange 66′, which is slanted relative to the central optic. More particularly, a normal N1 to an edge surface ES1 of the central optic is substantially orthogonal to an optical axis OA of the IOL whereas a normal N2 to an edge surface ES2 of the flange forms an angle θ relative to the optical axis. The flange can be configured to inhibit dysphotopsia, e.g., in a manner discussed above. Further, in some implementation of this or other embodiments, the thickness of the flange can be less than the minimum (or the average) thickness of the central optic (e.g., by a factor of about 5).

Although in the above embodiments, the IOL provides a single optical power, in other embodiments, it can include a diffractive structure so as to provide both a far-focus optical power as well as a near-focus power. By way of example, with reference to FIGS. 13A and 13B, an IOL 98 in accordance with one such embodiment includes an optic 100 formed of an anterior surface 102 and a posterior surface 104. The anterior surface 102 includes a central portion 102a that extends to a peripheral extension 102b, which partially surrounds the central portion. Similarly, the posterior surface 104 includes a central portion 104a that extends to a peripheral extension 104b, which partially surrounds the central portion. In many embodiments, the central portion of each of the anterior and the posterior surfaces is characterized by a radius relative to an optical axis OA in a range of about 2.5 mm to about 3.5 mm, while the peripheral extension of each of those surfaces can have a maximum radial distance from the optical axis in a range of about 3.5 mm to about 4.5 mm. The peripheral extension of at least one of the anterior or posterior surfaces can be configured, e.g., in a manner discussed above in connection with the previous embodiments, to inhibit the occurrence of dysphotopsia. By way of example, the peripheral extension of the anterior surface can be textured, or one or both surfaces can be opaque to visible radiation.

With continued reference to FIGS. 13A and 13B, the curvatures of the central portions of the anterior and posterior surfaces are selected such that the IOL would provide a desired far-focus optical power, e.g., in a range of about −15 D to about 34 D. A diffractive structure 140 that is disposed on the anterior surface provides a near focus optical power, e.g., in a range of about 1 D to about 4 D. In this embodiment, the diffractive structure 106 includes a plurality of diffractive zones 108 that are separated from one another by a plurality of steps that exhibit a decreasing height as a function of increasing distance from the optical axis—though in other embodiments the step heights can be uniform. In other words, in this embodiment, the step heights at the boundaries of the diffractive zones are “apodized” so as to modify the fraction of optical energy diffracted into the near and far foci as a function of aperture size (e.g., as the aperture size increases, more of the light energy is diffracted into the far focus). By way of example, the step height at each zone boundary can be defined in accordance with the following relation:

$$Step height = \frac{λ}{a ( {n}_{2} - {n}_{1} )} {f}_{apodize} \qquad Equation ( 1 )$$

wherein
λ denotes a design wavelength (e.g., 550 nm),
a denotes a parameter that can be adjusted to control diffraction efficiency associated with various orders, e.g., a can be selected to be 1.9;
n₂ denotes the index of refraction of the optic,
n₂ denotes the refractive index of a medium in which the lens is placed, and
ƒ_{apodize} represents a scaling function whose value decreases as a function of increasing radial distance from the intersection of the optical axis with the anterior surface of the lens. By way of example, the scaling function ƒ_{apodize} can be defined by the following relation:

$${f}_{apodize} = 1 - {( \frac{{r}_{i}}{{r}_{out}} )}^{2} . \qquad Equation ( 2 )$$

wherein
rᵢ denotes the radial distance of the i^{th} zone,
r^{out} denotes the outer radius of the last bifocal diffractive zone. Other apodization scaling functions can also be employed, such as those disclosed in a co-pending patent application entitled “Apodized Aspheric Diffractive Lenses,” filed Dec. 1, 2004 and having a Ser. No. 11/000770, which is herein incorporated by reference. In addition, further teachings regarding apodized diffractive lenses can be found in U.S. Pat. No. 5,699,142 entitled “Diffractive Multifocal Ophthalmic Lens,” which is herein incorporated by reference.

In this exemplary embodiment, the diffractive zones are in the form of annular regions, where the radial location of a zone boundary (rᵢ) is defined in accordance with the following relation:

rᵢ²=(2i+1)λƒ Equation (3)

wherein
i denotes the zone number (i=0 denotes the central zone),
rᵢ denotes the radial location of the ith zone,
λ denotes the design wavelength, and
ƒ denotes an add power.

A variety of IOL fabrication techniques known in the art, such as injection molding, can be employed to form IOLs according to the teachings of the invention.

Those having ordinary skill in the art will appreciate that various changes can be made to the above embodiments without departing from the scope of the invention.

What is claimed is:

1. An intraocular lens (IOL), comprising
an optic comprising a central portion and a peripheral extension that partially surrounds said central portion,
wherein, upon implantation of the IOL in a patient's eye, the optic forms an image of a field of view and the peripheral extension inhibits the perception of visual artifacts in a peripheral visual field of the patient.

2. The IOL of claim 1, wherein said peripheral extension inhibits peripheral light rays that enter the eye at large visual angles from forming a secondary peripheral image.

3. The IOL of claim 1, wherein said peripheral extension directs some light rays into a retinal shadow region between a secondary image formed by light rays entering the eye that miss the IOL and said image of the field of view generated by the IOL.

4. The IOL of claim 1, wherein said peripheral extension is adapted such that at least some light rays entering the eye's pupil at visual angles in a range of about 50 degrees to about 80 degrees are incident thereon.

5. The IOL of claim 1, wherein said central portion is rotationally symmetric about an optical axis of said optic and said peripheral extension is rotationally asymmetric about said axis.

6. The IOL of claim 4, wherein said peripheral extension is positioned in the eye so as to receive peripheral light rays entering the eye from the temporal side.

7. The IOL of claim 1, wherein said central portion has a maximum radial extension relative to an optical axis of said optic in a range of about 2 mm to about 3.5 mm and said peripheral extension has a maximum radial span relative to said axis in a range of about 2.5 mm to about 4.5 mm.

8. The IOL of claim 1, wherein said optic includes an anterior surface and a posterior surface, wherein a boundary of at least one of said surfaces exhibits a maximum radial distance from an optical axis of said optic that is greater than about 3.5 mm and a respective minimum radial distance that is less than about 3.1 mm.

9. The IOL of claim 1, wherein said optic is foldable so as to allow its insertion into the eye.

10. The IOL of claim 1, wherein said peripheral extension comprises at least one textured surface adapted to cause scattering of said peripheral light rays.

11. The IOL of claim 1, wherein said peripheral extension comprises at least one surface that is opaque to visible radiation.

12. The IOL of claim 1, wherein said peripheral extension comprises at least one curved surface adapted to redirect said peripheral light rays.

13. The IOL of claim 1, wherein said peripheral extension comprises any of a diffractive structure or a Fresnel lens.

14. An intraocular lens (IOL), comprising
an optic comprising an anterior surface and a posterior surface disposed about an optical axis, each of said surfaces comprising a central portion that is partially surrounded by a peripheral extension, said surfaces being adapted such that, when the IOL is implanted in a patient's eye, at least a portion of peripheral light rays entering the eye at large visual angles are incident on the peripheral extension of said anterior surface,
wherein at least one of said peripheral surface extensions is textured so as to scatter said peripheral light rays.

15. The IOL of claim 14, wherein the central portion of at least one of said surfaces has a maximum radial distance from said optical axis in a range of about 2.5 mm to about 3.5 mm and the respective peripheral extension of that surface has a maximum radial distance from said optical axis in a range of about 3.5 mm to about 4.5 mm.

16. The IOL of claim 15, wherein the peripheral extension of the anterior surface is textured.

17. The IOL of claim 14, wherein curvatures of said central surface portions are adapted to provide an optical power in a range of about −15 D to about 40 D.

18. The IOL of claim 15, further comprising a diffractive structure disposed on the central portion of one of said surfaces so to provide a far-focus and a near-focus optical power.

19. The IOL of claim 18, wherein said IOL has a near-focus power is in a range of about 1 D to about 4 D.

20. An intraocular lens (IOL), comprising
an optic comprising an anterior surface and a posterior surface, said optic being characterized by a central portion and a peripheral extension, said peripheral extension partially surrounding said central portion so as to receive peripheral light rays entering a patient's eye in which the IOL is implanted at large visual angles,
wherein the peripheral extension comprises at least one surface that is opaque to visible radiation.

21. The IOL of claim 20, wherein said opaque extension surface inhibits said peripheral light rays from reaching the patient's retina.

22. The IOL of claim 20, wherein said opaque extension surface comprises a portion of said anterior surface.

23. The IOL of claim 19, wherein said optic provides an optical power in a range of about −10 D to about 40 D.

24. The IOL of claim 23, further comprising a diffractive structure disposed on at least one of said anterior or posterior surfaces so as to provide a far-focus and a near-focus optical power.

25. An intraocular lens (IOL), comprising
an optic comprising a central portion surrounded partially by a peripheral extension,
wherein upon implantation of the IOL in a patient's eye, the optic forms an image of a field of view on the patient's retina and the peripheral extension inhibits formation of a secondary image by peripheral light rays entering the eye at large visual angles.

26. The IOL of claim 25, wherein said optic comprises an anterior surface and a posterior surface disposed about an optical axis, wherein each of said surfaces is characterized by a central portion extending to a peripheral extension.

27. The IOL of claim 26, wherein the central portion of each of said surfaces is rotationally symmetric about said optical axis and the respective peripheral extension is rotationally asymmetric about said axis.

28. The IOL of claim 27, wherein each of said surfaces is characterized by two orthogonal meridians, wherein one meridian exhibits a radial extension in a range of about 2.5 mm to about 3.5 mm from said axis and the other exhibits a radial extension in a range of about 3.5 mm to about 4.5 mm from said axis.

29. The IOL of claim 25, wherein at least one of said surfaces exhibits an asphericity characterized by a conic constant in a range of about −10 to about −100.

30. The IOL of claim 26, further comprising a diffractive structure disposed on at least one of said surfaces such that the optic provides a far-focus optical power and a near-focus optical power.

31. The IOL of claim 24, wherein said optic is foldable so as to facilitate its insertion in the eye.

32. An intraocular lens (IOL), comprising
an optic disposed about an optical axis, said optic providing an optical power for generating an image of a field of view on the retina of a patient's eye in which the IOL is implanted, and
an optical flange at least partially surrounding said optic, said flange being adapted to receive peripheral light rays entering the patient's eye at large visual angles and to inhibit said peripheral rays from forming a secondary retinal image.

33. The IOL of claim 33, wherein said optic has a maximum radial extension in a range of about 2 mm to about 3.5 mm relative to said optical axis and said optical flange has a maximum radial extension in a range of about 2.5 mm to about 4.5 mm from said axis.

34. The IOL of claim 32, wherein said optical flange includes at least one surface that is opaque to visible light.

35. The IOL of claim 32, wherein said optical flange includes at least one surface that is textured so as to substantially scatter light incident thereon.

36. The IOL of claim 35, wherein said textured surface is characterized by a plurality of surface undulations having amplitudes comparable to wavelengths of visible light.

37. The IOL of claim 36, wherein said surface undulations exhibit physical surface amplitudes in a range of about 0.2 microns to about 2 microns.

38. The IOL of claim 35, wherein said textured surface comprises an anterior surface of said flange.

39. The IOL of claim 32, wherein said optic and said flange are foldable to facilitate placement of the IOL in the patient's eye.

40. The IOL of claim 32, wherein said optical flange provides an optical power less than an optical power of said optic.

41. The IOL of claim 40, wherein the optical power of said flange is less than that of the optic by a factor in a range of about 25% to about 75%.

42. The IOL of claim 32, wherein said flange includes any of a diffractive structure or a Fresnel lens.

43. A method of correcting vision, comprising
providing an optic for implantation in a patient's eye, said optic comprising a central portion partially surrounded by a peripheral extension,
implanting said optic in a patient's eye,
wherein once implanted said optic forming an image of a field of view with the peripheral extension inhibiting dysphotopsia.

44. An intraocular lens (IOL), comprising
an optic comprising a central portion and a peripheral extension that partially surrounds said central portion,
wherein said peripheral extension is adapted to inhibit dysphotopsia once the IOL is implanted in a patient's eye.

45. The IOL of claim 44, wherein said peripheral extension inhibits dysphotopsia by capturing peripheral light rays entering the eye at large visual angles.

46. The IOL of claim 44, wherein said peripheral extension inhibits dysphotopsia by directing at least some light rays incident thereon to a shadow region between an image formed by the IOL and a second peripheral image formed by rays entering the eye that miss the IOL.
